(19) United States
(12) Patent Application Publication
(10) Pub. No.: US 2022/0047184 A1
(43) Pub. Date: Feb. 17, 2022

(54) BODY NOISE-BASED HEALTH MONITORING

(71) Applicant: Cochlear Limited, Macquarie University, NSW (AU)

(72) Inventor: Riaan Rottier, Eastwood, NSW (AU)

(21) Appl. No.: 17/413,727

(22) PCT Filed: Jun. 17, 2020
(86) PCT No.: PCT/IB2020/055652

§ 371 Date: Jun. 14, 2021

Related U.S. Application Data
(60) Provisional application No. 62/866,045, filed on Jun. 25, 2019.

(51) Int. Cl. A61B 5/11; A61B 5/00; A61B 7/04; A61B 7/02

(52) U.S. Cl. CPC A61B 5/1118; A61B 5/4803; A61B 5/686; A61B 2562/0204; A61B 7/04; A61B 7/023; A61B 5/7264

(57) ABSTRACT

Presented herein are techniques that can be used to track/monitor the health/well-being of an individual, such as person of an implantable medical prosthesis system, in a manner that protects the individual's privacy. In particular, a system in accordance with embodiments presented herein comprises one or more sensors configured to detect signals that may comprise one or more of external acoustic sounds and/or body noises (i.e., sounds originating from within the body of the person). The outputs from one or more sensors are analyzed to identify and categorize the individual's body noises present in the detected signals. The body noises are categorized in terms of the person's current/real-time activity.

### BACKGROUND

### Field of the Invention

The present invention relates generally to body noise-based health monitoring in medical prosthesis systems.

### Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In one aspect, a system is provided. The system comprises: at least a first sensor configured to be implanted in or worn on a person, wherein the at least first sensor is configured to detect body noises of the person; and an activity classifier configured to determine, based at least on the body noises, an activity classification of the person's current activity.

In another aspect, a method is provided. The method comprises: detecting, over a first period of time, signals at first and second sensors of a body noise-based health monitoring system, wherein the signals detected at one or more of the first and second sensors include body noises of a person and acoustic sound signals; over the first period of time, determining, based at least on the body noises of the person, a first plurality of activity classifications for the person, wherein each of the first plurality of activity classifications indicates a real-time activity of the person at a time that an associated activity classification is generated; and storing the first plurality of activity classifications for the person.

In another aspect, a method is provided. The method comprises: detecting, at a first sensor configured to be implanted in or worn on a person, a plurality of body noises of the person; and generating, using the plurality of body noises, a plurality of activity classifications of the person, wherein each of the plurality of activity classifications indicates a real-time activity of the person at a when at least one of the plurality of body noises was detected.

### BRIEF DESCRIPTION OF THE DRAWINGS

Embodiments of the present invention are described herein in conjunction with the accompanying drawings, in which:

FIG. 1A is a block diagram of a body noise-based health monitoring system, in accordance with certain embodiments presented herein;

FIG. 1B is a block diagram of another body noise-based health monitoring system, in accordance with certain embodiments presented herein;

FIG. 2 is a table illustrating activity classifications generated by a body noise-based health monitoring system, in accordance with certain embodiments presented herein;

FIG. 3 is a diagram illustrating an example graphical display as determined from a recipient body noises and external acoustic sounds, in accordance with certain embodiments presented herein;

FIG. 4 is a block diagram of another body noise-based health monitoring system, in accordance with certain embodiments presented herein;

FIG. 5A is a schematic diagram illustrating an implantable auditory prosthesis in accordance with embodiments presented herein implanted in a recipient;

FIG. 5B is a block diagram of the implantable auditory prosthesis of FIG. 5A;

FIG. 6 is a schematic diagram of a spinal cord stimulator, in accordance with certain embodiments presented herein;

FIG. 7 is a flowchart of a method, in accordance with certain embodiments presented herein; and

FIG. 8 is a flowchart of a method, in accordance with certain embodiments presented herein.

### DETAILED DESCRIPTION

There are certain individuals who have the ability to live independently, but have increased risk for disease, injury, incapacitation, etc. For example, segments of the world population are aging at a rapid rate and it is desirable to enable this aging population to live independently for as long as possible. Similarly, certain individuals suffering from disabilities, Down syndrome, autism, and/or other disorders have the ability to live or work independently from any caregivers. However, with increased age, disabilities, disorders, and/or other impairments also comes an increased risk of disease, injury, incapacitation, or some other potentially life-threatening health event.

It may be desirable, comforting, or medically necessary to monitor the health/well-being of individuals with increased risk of disease, injury, incapacitation, etc. Current approaches to such monitoring use, for example, cameras or multiple sensors placed within an individual's home (e.g., sensors fitted to the floor of every room, cupboard, etc. monitoring utility consumption and along with smart scales). However, these conventional monitoring approaches are inferential, complex, invasive, and deprive the individual of his/her privacy and independence (i.e., multiple cameras and sensors would need to be placed and would rely on inference to make assumptions, such as determining that a person spent time in the kitchen, opened the fridge and some cupboards, allowing the inference that a meal was consumed). As a result, there is a need to enable the monitoring of the health/well-being of individuals in a non-intrusive manner.

Presented herein are techniques that can be used to track/monitor the health/well-being of an individual, such as recipient of an implantable medical prosthesis system, in a manner that protects the individual's privacy. In particular, a system in accordance with embodiments presented herein comprises at least one sensor configured to detect at least body noises (i.e., sounds induced/originated by the body of the recipient that are propagated primarily as vibration within the recipient's bone, tissue, etc.). The system is configured to categorize the body noises in terms of the recipient's current/real-time activity.

More specifically, a system in accordance with embodiments presented herein is configured to monitor the individual's body noises and determine an activity classification for the recipient based thereon (e.g., determine the “class” or “category” of the individual's real-time actions, movements, non-movement, behavior, etc. based on the detected body noise). That is, the detected body noises, and potentially associated (simultaneously received) acoustic sound signals, can be associated with everyday activities and common bodily functions, such as a heartbeat, breathing, swallowing, chewing, talking, drinking, brushing teeth, shaving, walking, scratching moving the head against various surfaces (sleeping, driving), etc. The recipient's activity classifications can be logged, over time, and then analyzed to evaluate the health of the recipient (e.g., provide confidence of good health or detect health changes that might require intervention or further investigation, etc.).

Merely for ease of illustration, the techniques presented herein are primarily described with reference to “stand-alone” body noise-based health monitoring systems. As described further below, a stand-alone body noise-based health monitoring system is a system that is primarily configured to monitor the health/well-being of a person/individual, referred to herein as “recipient,” using the recipient's body noises. However, as detailed further below, the techniques presented herein may be implemented in a number of different manners such as, for example, in combination with different implantable medical prostheses. For example, the techniques presented herein may be used with or incorporated in cochlear implants or auditory prostheses, such as auditory brainstem stimulators, electro-acoustic hearing prostheses, acoustic hearing aids, bone conduction devices, middle ear prostheses, direct cochlear stimulators, bimodal hearing prostheses, etc. The techniques presented herein may also be used with balance prostheses (e.g., vestibular implants), retinal or other visual prosthesis/stimulators, occipital cortex implants, sensor systems, implantable pacemakers, drug delivery systems, defibrillators, catheters, seizure devices (e.g., devices for monitoring and/or treating epileptic events), sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc.

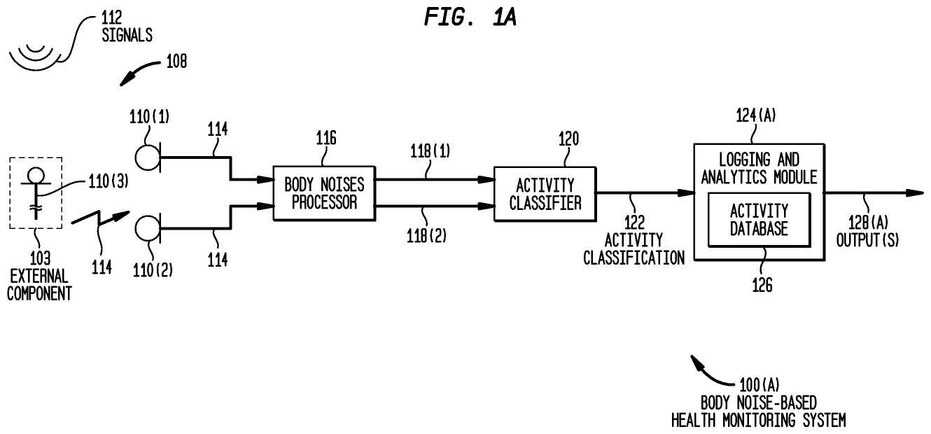

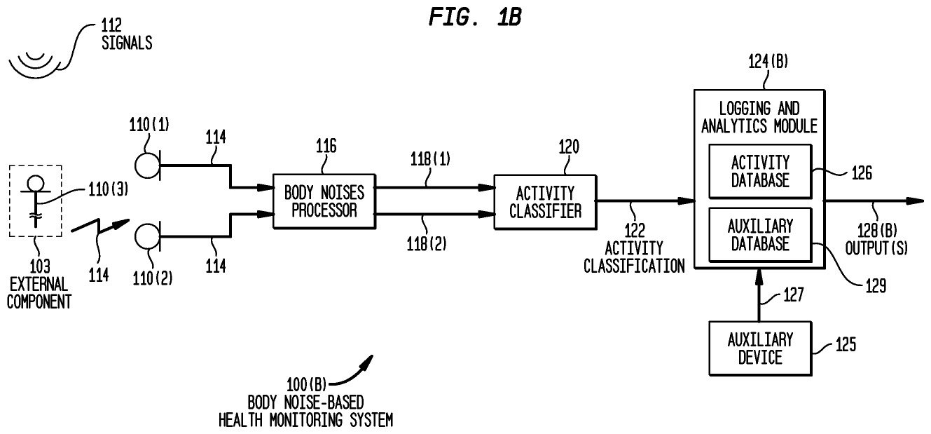

FIG. 1A is a functional block diagram of an exemplary of a body noise-based health monitoring system 100(A) in accordance with embodiments presented herein. As noted above, and as described further below, a body noise-based health monitoring system, such as body noise-based health monitoring system 100(A), is configured to track the health/well-being of a recipient (e.g., individual/person) of the system based-on (using) body noises of the recipient. As used herein, body noises (BNs) are sounds induced by the body that are propagated primarily as vibration within the recipient's bone, tissue, etc. (e.g., full spectrum of vibrations, including sub-acoustic and acoustic vibrations and potentially vibrations above 20 kilohertz (kHz)).

In accordance with embodiments presented herein, a body noise-based health monitoring system, such as system 100(A), includes at least one sensor configured to detect body noises. However, FIG. 1A illustrates a specific embodiment in which the body noise-based health monitoring system 100(A) includes the at least one sensor configured to detect body noises, as well as two additional (optional) sensors. As described further below, a first one of the additional sensors is used for separation of external sounds from body noises, and a second one of these additional sensors is used for separation of different internal body noises and, potentially, for some separation of external sounds. It is to be appreciated that the use of the two additional sensors is merely illustrative one of on example arrangement presented herein.

More specifically, in the specific example arrangement of FIG. 1A, the body noise-based health monitoring system 100(A) includes a first sensor 110(1), a second sensor 110(2), and a third sensor 110(3). Collectively, sensors 110(1), 110(2), and 110(3) are referred to herein as a “multi-channel sensor system” 108. In general, the sensors 110(1), 110(2), and 110(3) are configured to receive/detect input signals 112, which may include one or more of body noises (e.g., signal/vibrations originating from within the body of the recipient) and one or more of external acoustic sound signals associated with the body noises (e.g., sound signals originating outside of the body that are received at the same time as the body noises). The sensors forming a multi-channel sensor system in accordance with presented herein can take a variety of different forms, such as microphones, accelerometers, etc. However, merely for ease of illustration, FIG. 1A illustrates a multi-channel sensor system 108 where sensor 110(1) is a microphone, sensor 110(2) is an accelerometer, and sensor 110(3) is another microphone.

The sensors 110(1), 110(2), and 110(3) are configured to detect/receive signals 112, which include body noises and/or external sounds (i.e., signals 112 may be acoustic signals, vibrations, etc., that originate from within or outside of the recipient's body). In general, microphone 110(1) is configured to detect body noises forming part of the signals 112. The accelerometer 110(2) detects vibrations and the signals detected thereby are used for separation of different internal body noises forming part of signals 112. The signals detected by accelerometer 110(2) may also potentially be used for some separation of external sounds from the body noises within signals 112. The microphone 110(3) is configured to detect external sounds forming part of the signals 112 and, as such, the signals detected thereby are used for separation of external sounds from body noises. microphone 110(1) microphone 110(1) accelerometer 110(2) microphone 110(1) accelerometer 110(2) microphone 110(1) accelerometer 110(2)

As described elsewhere herein, the sensors 110(1), 110(2), and 110(3) can have different arrangements, locations, etc. However, for purposes of illustration, sensor 110(3) (e.g., microphone) is shown in FIG. 1A as part of an external component 103. In an alternative arrangement, the sensor 110(3) may be implanted in the recipient such that the sensor 110(3) is well isolated from body noises (e.g., a tube microphone).

Although, for ease of description, embodiments presented herein are primarily described with reference to the use of a microphone 110(1), accelerometer 110(2), and a microphone 110(3), it is to be appreciated that these specific implementations are non-limiting. As such, embodiments of the present invention may be used with different types and combinations of sensors having various locations, configurations, etc. It also appreciated that the multi-channel sensor system 108 could include additional or fewer sensors.

Returning to the example of FIG. 1A, the multi-channel sensor system 108 (i.e., microphone 110(1), accelerometer 110(2), and microphone 110(3)) is configured to detect/receive the input signals 112 (sounds/vibrations from external acoustic sounds and/or body noises) and convert the detected input signals 112 into electrical signals 114, which are provided to a body noises processor 116. The body noises processor 116, which may include one or more signal processors, is configured to execute signal processing operations to convert the electrical signals 114 into processed signals that represent the detected signals. As a result of the signal processing operations, the body noises processor 116 outputs a first processed signal 118(1) representing features of the detected body noises and a second processed signal 118(2) representing features of the detected acoustic sound signals. That is, the body noises processor 116 extracts and preserves features of the body noise features and acoustic sound signals, where the features are represented in signals 118(1) and 118(2), which are then provided (e.g., via a wired or wireless connection) to an activity classifier 122.

In certain examples, the body noises processor 116 is configured to perform one or more privacy protection operations to protect the privacy of the recipient. For example, the body noises processor 116 may be configured to ensure that it is not possible for any captured speech to be reconstructed from the features (e.g., discontinuously recording the received audio inputs). In certain examples, the body noises processor 116 and/or the logging and analytics module 124(A) (described further below) is/are configured to execute privacy protection operations that block the output of certain classification categories that the recipient would prefer to keep private. This type of privacy protection could be enabled at the recording stage or the classification stage (e.g., eliminating/omitting certain classifications that are not to be shared). Alternatively, the certain classification categories can still be generated as described further below, but shown privately to the recipient only (e.g., not shared with others).

In addition or alternatively, a federated learning approach could be used to protect a recipient's privacy. Use of an example federated learning approach is described in greater detail below.

Returning to the example of FIG. 1A, the activity classifier 120 receives the signals 118(1) and 118(2) generated by the body noise processor 116. The activity classifier 120 is configured to monitor the signals 118(1) and 118(2) and perform an analysis thereon to determine the “class” or “category” of the detected body noises in terms of recipient's real-time activity, behavior, or actions (collectively and generally “activity”). That is, the activity classifier 120 is configured to use the signal features (i.e., characteristics) extracted from the signals 112 captured by the multi-channel sensor system 108 to generate a real-time classification of the detected body noises. A real-time activity classification determined by the activity classifier 120 is generally represented in FIG. 1A by arrow 122 and sometimes referred to herein as “activity classification” or “activity class” 122.

As noted, the activity classifier 120 is configured to use the both the extracted body noise features, as well as the extracted acoustic sound features, to generate the activity classification 122 associated current/real-time activity of the recipient (i.e., the activity of the recipient at the time the body noises within signals 112 is detected). Although the activity classification 122 corresponds to the body noises, the acoustic sound signals detected at the time of the body noises provide context to the body noises. As such, the activity classification 122 is based not only on the body noises, but also on any external acoustic sound signals that are detected at the time the body noises are detected.

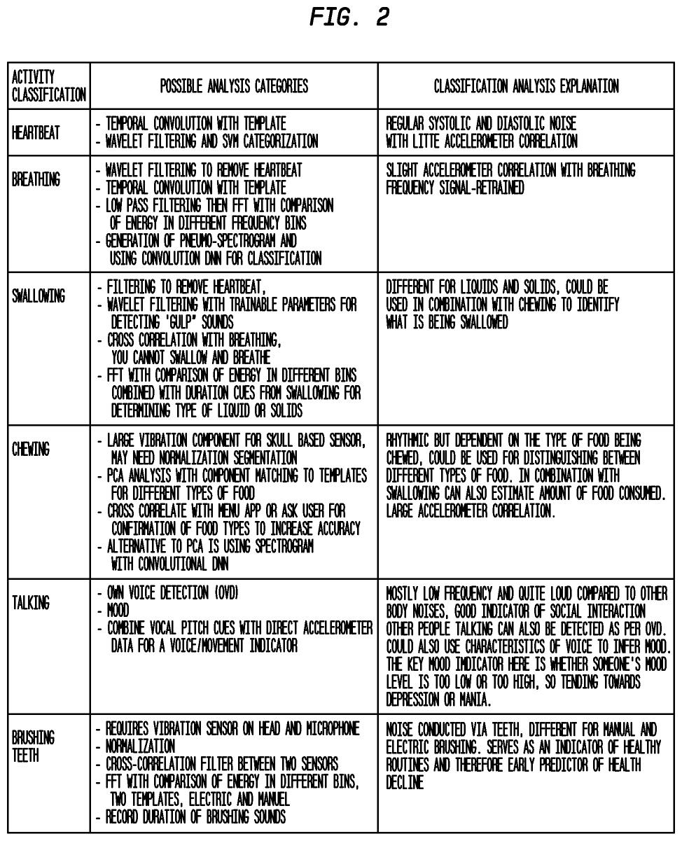

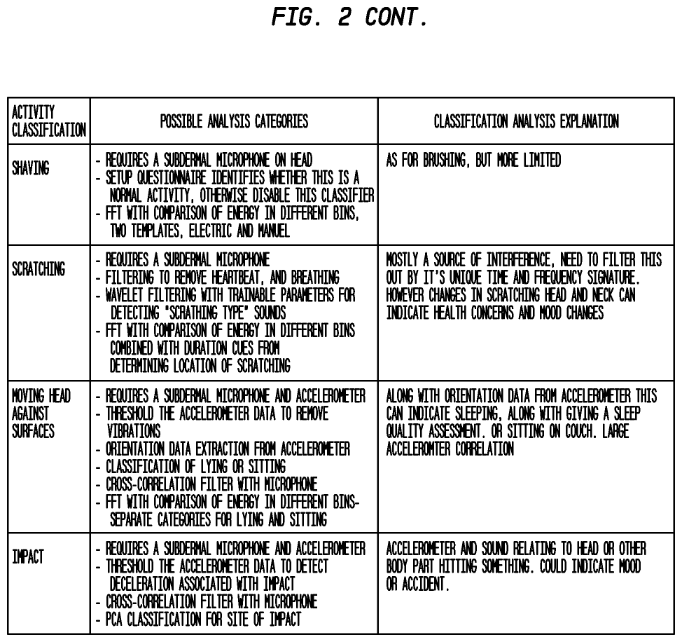

Table 1, which is shown in FIG. 2, illustrates several example activity classifications that can be made by an activity classifier, such as activity classifier 120, in accordance with certain embodiments presented herein. Along with the example classifications, Table 1 also includes an explanation as to basis for the example activity classifications. It is to be appreciated that the activity classifications shown in Table 1, as well as the resulting explanations, are merely illustrative of several activity classifications that can be generated in accordance with embodiments presented. As such, Table 1 should be viewed as a non-exhaustive list of activity classifications that can be generated by an activity classifier in accordance with certain embodiments presented herein.

As shown in Table 1, the activity classifications made by activity classifier 120 are not necessarily mutually exclusive (i.e., several activities may be detected at the same time). In certain such examples, the activity classifications are treated as a multi-label problem where the system predicts the probability of the input containing each of the target categories and then establishes a threshold for when/how the activity classifier decides that the probability is high enough to determine that an activity is present. Alternatively, the system can include a multiplicity of classifiers that trained from the raw signals, which are then combined as the outputs.

The above examples are merely illustrative. In general, the activity classifier 120 may analyze the signal features extracted from the input signals 112 captured by the multi-channel sensor system 108, as represented in signals 118(1) and 118(2), in a number of different manners to determine the activity classification 122. For example, as shown in Table 1, the activity classifier 120 may be configured to perform a time domain and/or frequency domain analysis of the signal features extracted from the input signals 112 to determine the activity classification 122. The activity classifier 120 may also or alternatively perform comparisons or correlations of the signal features extracted from the input signals 112 (e.g., in terms of level, timing, etc.). In certain examples, the activity classifier 120 is configured to perform a multi-dimensional analysis of the signal features extracted from the signals 112. As a result, the features extracted from the input signals 112 may take different forms and can include time information, signal levels, frequency, measures regarding the static and/or dynamic nature of the signals, etc. The activity classifier 120 operates to determine the category of for the recipient's body noises (i.e., activity classification) using a type of decision structure (e.g., machine learning algorithm, decision tree, and/or other structures that operate based on individual extracted characteristics from the input signals). Further details regarding example machine learning approaches to this classification are provided below.

In particular, a machine learning algorithm may be trained to perform the activity classification using samples of labelled noise in accordance with techniques such as random forest ensembles, deep neural networks (DNNs) or Support Vector Machines. The classification categories may be customized to the specific recipient where, for example, the normal breathing sound can vary from recipient to recipient and can also depend on additional factors (e.g., health, whether the recipient is lying down, physical activity, etc.). The techniques presented herein may also apply one shot learning to customize a machine learning algorithm to a specific recipient and then use prompts through an interface to classify the additional factors. In certain examples, the signature of a specific activity may be similar for all recipients. In general, the parameters of the expert algorithm or the weights/parameters of the machine learning algorithm would be updated through the cloud to, for example, add new categories, increase accuracy, adapt to new implant capabilities, provide updates, integrate more sensors in the current classification, etc. As described elsewhere herein, for customization the system could use inputs from other systems used to track activity (e.g., body-worn fitness trackers or other wearable devices, one of the described monitoring systems, etc.).

As shown in FIG. 1A, the activity classification 122 generated by the activity classifier 120 is provided to a logging and analytics module 124(A). In general, the logging and analytics module 124(A) is configured to log (e.g., store) the activity classifications 122 generated for the recipient over a period time (e.g., one or days, one or more weeks, etc.). In accordance with embodiments presented herein, the activity classifications 122 are logged with time information (e.g., time stamps) that indicate, for example, a time-of-day (ToD) and/or date when a particular activity classification is generated. The activity classifications 122 may be provided to the logging and analytics module 124(A) continually, at certain intervals or periodically, only upon the determination of an activity classification change, or in another manner. As a result, the logging and analytics module 124(A) generates/populates, over time, an activity database 126 (i.e., the log of the activity classifications 122 over time). That is, the activity database 126 is populated with the activity classifications 122 in relation to the time information.

At least initially, the activity database 126 may be analyzed to create a profile of normal habits and behaviors for the specific recipient, sometimes referred to herein as one or more “baseline behavior patterns” for the recipient. As used herein, a behavior pattern is the typical activities performed by a recipient during one or more time periods. In certain examples, a recipient's behavior pattern includes an indication of a length of time for which the recipient engages in the activities, the time-of-day the recipient starts/begins the activities, or other time information associated with the activities.

Subsequently, the activity database 126 may be analyzed to determine one or more deviations or changes from the baseline behavior patterns (i.e., changes to the normal habits and behaviors for the specific recipient). The activity database 126 analysis may result in the generation of one or more outputs 128(A). These outputs 128(A) may take a number of different forms and, with suitable de-identification as described elsewhere herein, can be provided to a user, such as the recipient, family members, health professionals, etc. for use in monitoring the recipient's health/well-being.

For example, in certain embodiments, the activity classifications 122 for the recipient over a first period of time may be used to generate one or more baseline behavior patterns for the recipient. The health of the recipient may then be monitored using these one or more baseline behavior patterns. For example, a plurality of activity classifications 122 for the recipient determined over a second period of time may be used to generate one or more current or real-time behavior patterns for the recipient (i.e., the habits and behaviors for the specific recipient during the second period of time, which is different from the first period of time). The one or more current behavior patterns may be analyzed relative to the one or more baseline behavior patterns (e.g. compared to) to detect one or more differences between the current behavior patterns and the one or more baseline behavior patterns. As described further below, if certain one or more differences between the one or more current behavior patterns and the one or more baseline behavior patterns are detected, the system 100(A) can generate one or more messages configured to initiate or elicit a remedial action.

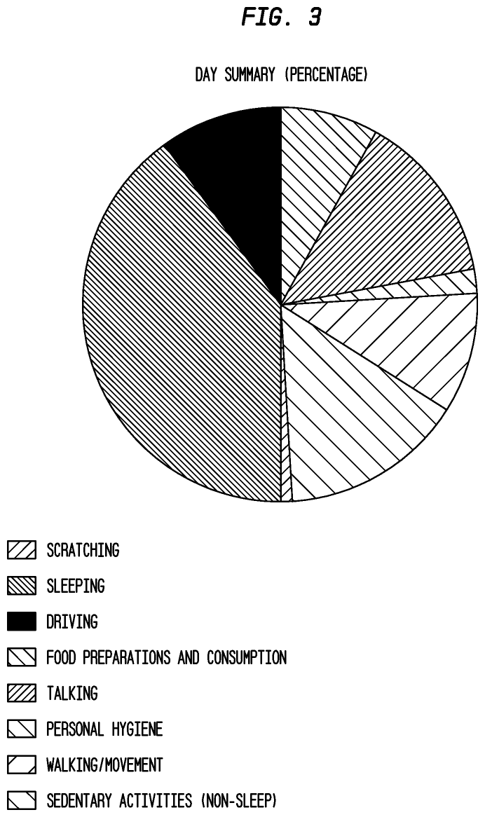

In certain embodiments, the outputs 128(A) may be used to generate health monitoring information (e.g., text, graphical displays, etc.) for display via a computing device. For example, FIG. 3 illustrates an example graphical display (e.g. pie chart) summarizing a recipient's daily activities, as determined from the recipient body noises and external acoustic sounds detected at the recipient's body noise-based health monitoring system. In particular, FIG. 3 illustrates the percentage of the time that the recipient spent engaged in the particular activity throughout the course of the selected day. FIG. 3 illustrates one example of a daily chart that can show daily routines for comparison to a baseline (e.g., for deviations from normal routines). The example graphical display of FIG. 3 is merely illustrative and it is to be appreciated that health monitoring information in accordance with embodiments presented herein can have a number of different forms.

As noted above, in certain embodiments, the outputs 128(A) could comprise messages, alerts, prompts, etc. (collectively and generally referred to herein as messages) that are configured to initiate or elicit a remedial action (e.g., a message to the recipient to increase their fluid intake or warn them that their level of physical activity had been declining, a notification to a family member of a potential health issue, etc.). That is, the activity database 126 could be monitored or analyzed (e.g., using one or more additional machine learning algorithms) in order to generate alerts if the recipient's behavior patterns deviate from one or more baseline behavior patterns in a concerning way. In general, the analysis is not intended to primarily detect specific health events, although it is envisaged that specific health events (e.g., cardiac arrest or impending stroke) can be predicted or detected from the activity classifications 122 within activity database 126. Instead, the system attempts to detect the patterns that may be of concern to family member who may not be physically with the recipient and act as a prompt for intervention (e.g., determine the recipient is not eating or drinking as much as before, detect changes in sleep patterns, etc.). As such, the certain embodiments, the outputs 128(A) may represent information identifying changes to the recipient's lifestyle (e.g., indicated in comparison to the baseline or another metric).

For example, the body noise-based health monitoring system 100(A) may be configured to use the recipient's body noises to determine when the recipient is sleeping (e.g., categorize the recipient's activity as “sleeping”) and whether the recipient is moving (e.g., categorize the recipient's activity as “movement”) or moving in specific manner (e.g., sub-categorize the “movement” in some manner). At some point in time, the body noise-based health monitoring system 100(A) detects that there has been a change in the recipient's “sleeping” and “movement” activities during typical sleeping hours (e.g., the body noises associated with the recipient's typical sleeping pattern has changed and the recipient has been rolling around and/or awake for several nights in a row). In addition, the body noise-based health monitoring system 100(A) also detects that the person had been eating less (e.g., less time periods in a “chewing” activity classification) and not moving around as much (e.g., less time in a “walking” activity classification). This combination or events, and the fact that it persists over few days, could trigger the system 100(A) to issue an alert to the recipient's physician to check in with the recipient.

The recipient's logged activity classifications (e.g., activity database 126) can be stored in a number of different manners in a number of different locations. In certain examples, the activity classifications may be stored locally (e.g., a personal computing device), while in other embodiments the activity classifications may be stored in private cloud storage.

As noted, FIG. 1A illustrates a body noise processor 116, activity classifier 120, and logging and analytics module 124(A). Each of the body noise processor 116, activity classifier 120, and logging and analytics module 124(A) may be formed by one or more processors (e.g., one or more Digital Signal Processors (DSPs), one or more uC cores, etc.), firmware, software, etc. arranged to perform operations described herein. That is, the body noise processor 116, activity classifier 120, and logging and analytics module 124(A) may each be implemented as firmware elements, partially or fully implemented with digital logic gates in one or more application-specific integrated circuits (ASICs), partially in software, etc.

Also as noted, FIG. 1A illustrates an embodiment with a microphone 110(1), an accelerometer 110(2), and a microphone 110(3). As noted, the use of these three sensors is merely illustrative and that embodiments of the present invention may be used with different types and combinations of sensors having various locations, configurations, etc. It also to be appreciated that the multi-channel sensor system 108 could include different numbers of sensors.

In summary, FIG. 1A illustrates an arrangement configured to detect and classify a recipient's body noises in terms of the recipient's real-time activity. That is, the body noises are associated with everyday activities and common bodily functions like heartbeat, breathing, swallowing, chewing, talking, drinking, brushing teeth, shaving, walking, scratching and moving the head against various surfaces (sleeping, driving). The recipient's real-time activity is logged, over time, and used for lifestyle and health monitoring.

In the embodiment of FIG. 1A, the logging and analytics module 124(A) generates the one or more outputs 128(A) based on the activity classifications 122. It is to be appreciated that the one or more outputs 128(A) are not necessarily used in isolation, but instead can be combined with that of other health applications to gather further insights into the recipient's health and well-being. Moreover, in certain examples, the one or more outputs 128(A) themselves may be generated based on the activity classifications 122 as well as additional information. One example of such arrangement is shown in FIG. 1B.

More specifically, FIG. 1B is a block diagram of a body noise-based health monitoring system 100(B), in accordance with embodiments present herein. The body noise-based health monitoring system 100(B) is similar to body noise-based health monitoring system 100(A) of FIG. 1A in that includes the multi-channel sensor system 108, body noises processor 116, and activity classifier 120 that are used to generate activity classifications 122. The body noise-based health monitoring system 100(B) also comprises a logging and analytics module 124(B) and one or more auxiliary devices 125.

The one or more auxiliary devices 125 may include, for example, various types of sensors, transducers, monitoring systems, etc. The one or more auxiliary devices 125 are configured to generate auxiliary health inputs 127 that are provided to the logging and analytics module 124(B) (i.e., inputs generated from signals other than body noise and/or sound signals). Therefore, as shown in FIG. 1B, the logging and analytics module 124(B) receives both the activity classifications 122 generated by the activity classifier 120, as well as the auxiliary health inputs 127 generated by the one or more auxiliary devices 125.

Similar to the embodiment of FIG. 1A, the logging and analytics module 124(B) is configured to generate/populate, over time, an activity database 126 using the activity classifications 122 (i.e., the log of the activity classifications 122 over time). Also similar to FIG. 1A, the activity classifications 122 are logged with time information (e.g., time stamps) that indicate, for example, a time-of-day (ToD) and/or date when a particular activity classification is generated, where the activity classifications 122 are received continually, at certain intervals or periodically, only upon the determination of an activity classification change, or in another manner. As a result, the activity database 126 is populated with the activity classifications 122 in relation to the time information.

In FIG. 1B, the logging and analytics module 124(B) is also configured to generate/populate, over time, one or more auxiliary databases 129 using the auxiliary health inputs 127 received from the one or more auxiliary devices 125. In particular, the auxiliary health inputs 127 may be are logged with time information (e.g., time stamps) that indicate, for example, a time-of-day (ToD) and/or date when a particular auxiliary input is generated. The auxiliary health inputs 127 may be provided to the logging and analytics module 124(B) continually, at certain intervals or periodically, only upon the determination of a particular event, or in another manner. As a result, the one or more auxiliary databases 129 may be populated with the auxiliary health inputs 127 in relation to the time information.

Similar to the embodiments of FIG. 1A, the activity database 126 and the one or more auxiliary databases 129 may be analyzed to create a profile of normal habits and activities for the specific recipient. The activity database 126 and the one or more auxiliary databases 129 may also be analyzed and used to generate one or more outputs 128(B). Similar to the outputs 128(A) described with reference to FIG. 1A, the outputs 128(B) may take a number of different forms and, with suitable de-identification as described elsewhere herein, can be provided to a user, such as the recipient, family members, health professionals, etc. for use in monitoring the recipient's behavior and well-being. For example, the outputs 128(B) may be used to generate lifestyle information (e.g., text, graphical displays, etc.) for display via a computing device. In certain embodiments, the outputs 128(B) could comprise messages that are configured to initiate or elicit a remedial action (e.g., a message to the recipient to increase their fluid intake or warn them that their level of physical activity had been declining, a notification to a family member of a potential health issue, etc.)

As noted, the one or more auxiliary devices 125 may include various types of sensors, transducers, monitoring systems, etc. For example, in one arrangement the auxiliary device 125 may comprise a health monitor, such as a temperature tracker, heartrate monitor, blood pressure sensor configured to generate blood pressure measurements. These auxiliary health inputs can be logged and correlated with the activity classifications 122 to monitor the health and well-being of the recipient (e.g., correlate activities like eating with health effects like gaining or loosing weights). Certain recipient activities, together with a specific auxiliary health inputs, may be used to predict the level of health of an aging recipient and alert family members when something changes in a way that may require intervention.

In another example, the auxiliary device 125 may comprise a body-worn fitness tracker configured to track certain recipient's activities or activity levels. Collectivity, the activity information from a fitness tracker and the activity classifications 122 may be used to determine additional lifestyle information, such as certain (e.g., walking while eating/talking, etc.).

FIGS. 1A and 1B generally illustrate the components/elements of example body noise-based health monitoring systems in accordance with several embodiments presented herein. However, FIGS. 1A and 1B have been generally described without making reference to physical locations of the various components of the example body noise-based health monitoring systems or relative locations of the components of systems relative to one another. It is to be appreciated that the various components of body noise-based health monitoring systems may have a number of different relative arrangements and may be distributed across different devices. Different example arrangements for components of body noise-based health monitoring systems in accordance with embodiments presented herein are described below. However, it is to be appreciated that these examples are merely illustrative and that body noise-based health monitoring systems may be arranged in still other manners.

As noted, body noise-based health monitoring systems in accordance with embodiments presented herein include at least one sensor configured to capture the recipient's body noises. In certain embodiments, the body noise-based health monitoring systems may include additional sensors to, for example, capture external acoustic sound signals for subsequent use, as described above.

In certain embodiments presented herein in which a plurality of sensors are provided, all of the sensors are implanted within the recipient. In other embodiments, one or more of the plurality of sensors of a multi-channel sensor system may be implanted within the recipient, while the one or more of the sensors are non-implanted. The non-implanted sensors may be, for example, located in/on a head-worn component, located in a body-worn component, located in/on a mobile computing device carried by the recipient (e.g., mobile phone, remote control device, etc.), a wireless speaker or voice assistant device located in the environment (e.g., an assistant device in the bedroom, kitchen, living room etc.), etc. For example, the non-implanted sensors could, for example, sense movement in the living room that is correlated temporally with movement sounds from the body noise detector and infer the presence of the recipient in the kitchen which could classify activities as food preparation.

In still further embodiments, all of the plurality of sensors are non-implanted. However, in such embodiments, at least one of the plurality of sensors remains configured to detect the recipient's body noises. In one such example, a sound conductor (e.g., rigid rod, tube, etc.) is implanted within the recipient and firmly attached/coupled to bone of the recipient. At least one of the plurality of non-implanted sensors remains is, in turn, acoustically coupled to the sound conductor so as to sense vibration of the bone via the sound conductor. The acoustic coupling may be via a direct/physical connection, a coupling through the skin of the recipient, etc.

Also as noted above, the body noise-based health monitoring systems in accordance with embodiments presented herein include a body noises processor, an activity classifier, and a logging and analytics module. Again, these components can be distributed across one or a plurality of different physically separate devices.

For example, in certain embodiments, the body noises processor may be implemented in an implantable component configured to be implanted within the recipient (e.g., body noises processor is implanted with the plurality of sensors). Alternatively, the body noises processor may be implemented in a component configured to be worn by the recipient or a mobile computing device (e.g., mobile phone) carried by the recipient. As noted above, the body noises processor performs the first processing operations on the electrical signals generated by the sensors (e.g., microphone and accelerometer). Therefore, in general, the body noises processor may be implemented at a location proximate to (e.g., relatively close to) the sensors so that it can extract the body noise features and acoustic sound features.

As noted above, the activity classifier operates on the extracted body noise features and acoustic sound features obtained by the body noises processor, while the logging and analytics module operates using the activity classifications generated by the activity classifier. As such, and because the operations may require additional computing resources, the activity classifier and the logging and analytics module may be implemented separately from the body noises processor. For example, in certain embodiments, the activity classifier and the logging and analytics module may be implemented at a mobile computing device (e.g., mobile phone) carried by the recipient and/or at a computing system (e.g., local computer, one or more servers of a cloud computing system, etc.). In such embodiments, the extracted body noise features and acoustic sound features (e.g., signals 118(1) and 118(2) in FIG. 1A) are wirelessly transmitted from the component at which the body noises processor is implemented to the mobile computing device or computing system for the activity classification. If the activity classifier and logging and analytics module are implemented at different devices/systems, the activity classification is provided via a wired or wireless connection to the logging and analytics module

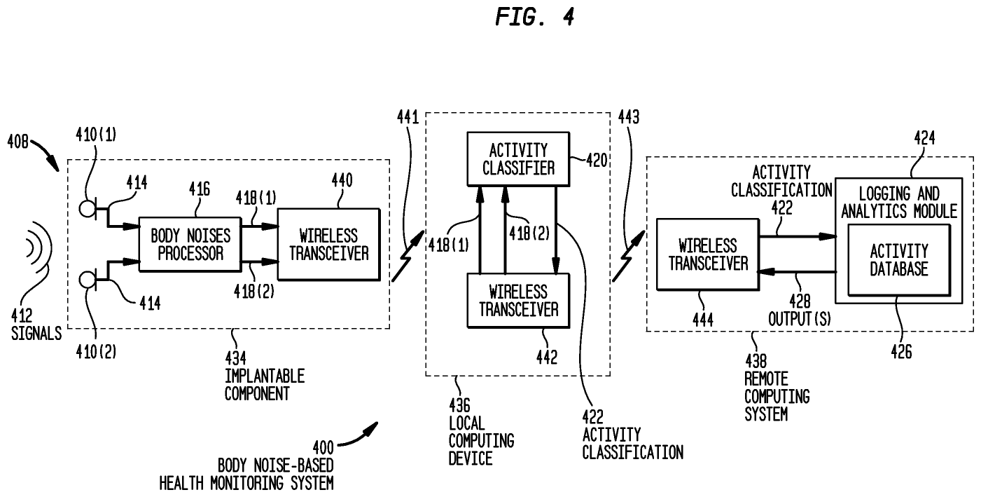

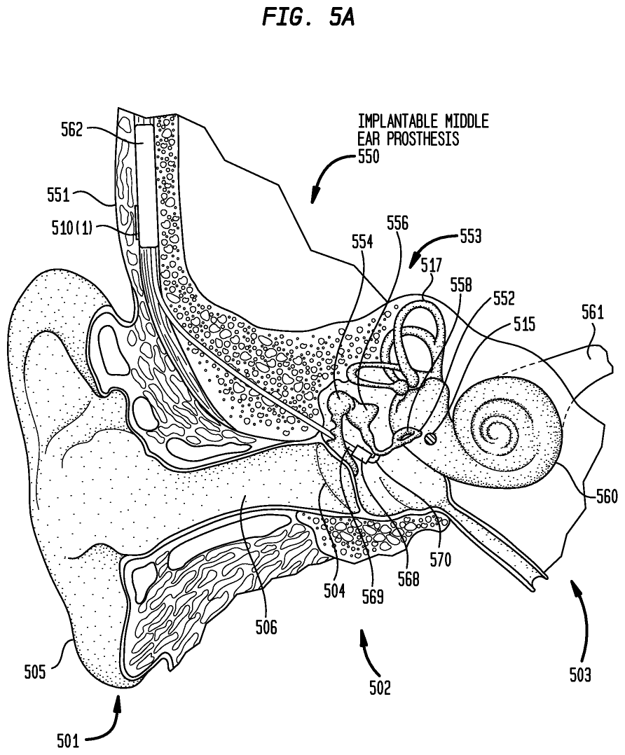

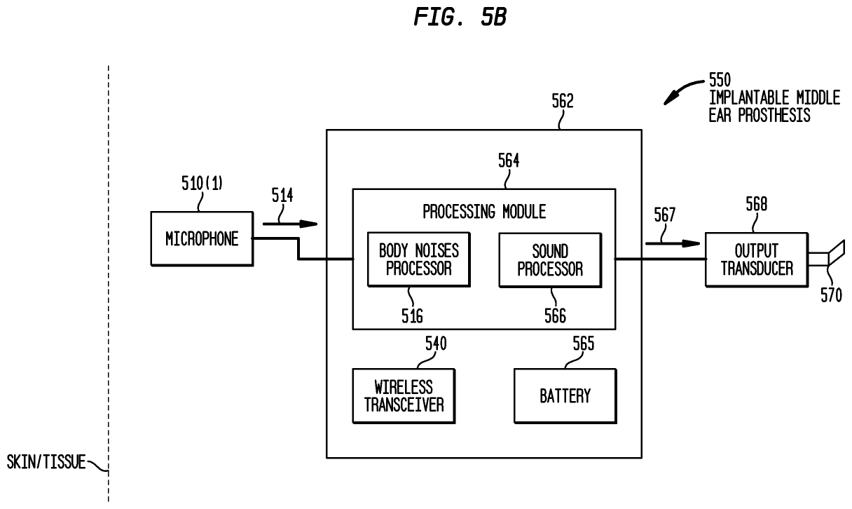

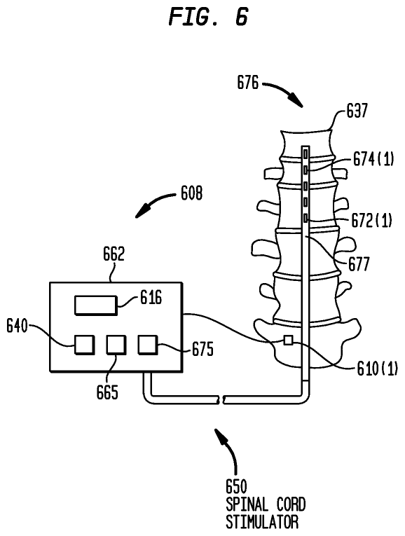

FIG. 4 illustrates a body-noise lifestyle tracking system that includes a stand-alone implantable component in accordance with embodiments presented herein, while FIGS. 5A, 5B, and 6 illustrate the incorporation of a body-noise lifestyle tracking system with different medical prostheses, in accordance with embodiments presented herein.

Referring first to FIG. 4, shown is an example body noise-based health monitoring system 400 in accordance with embodiments presented herein that comprises a stand-alone implantable component 434, a local computing device 436, and a remote computing system 438. The implantable component 434 is configured to be implanted within a recipient (e.g., under the recipient's skin/tissue), while the local computing device 426 is a physically separate device, such as a computer (e.g., laptop, desktop, tablet, etc.), mobile phone, etc.

The implantable component 434 is referred to as “stand-alone” component because, in this example, the implantable component 434 primarily operates to capture body noises for subsequent classification. However, as described below, this stand-alone configuration is merely illustrative and body noise-based health monitoring systems in accordance with embodiments presented herein may be incorporated with other types of medical prostheses.

The implantable component 434 includes a first sensor 410(1), a second sensor 410(2), a body noises processor 416, and a wireless transceiver 440. In this example, the first sensor 410(1) is a microphone, while the second sensor 410(2) is an accelerometer. Collectively, microphone 410(1) and the accelerometer 410(2) are referred to as a multi-channel sensor system 408.

The microphone 410(1) and the accelerometer 410(2) detect the input signals 412 (sounds/vibrations from external acoustic sounds and/or body noises) and convert the detected input signals 412 into electrical signals 414, which are provided to a body noises processor 416. The body noises processor 416, which may be similar to body noises processor 116 of FIGS. 1A and 1B, is configured to convert the electrical signals 414 into processed signals 418(1) and 418(2) that represent the detected signals. That is, the body noises processor 416 outputs a first processed signal 418(1) representing features of the detected body noises external acoustic sounds and a second processed signal 418(2) representing features of the detected external acoustic sounds (e.g., the body noises processor 416 extracts body noise features and acoustic sound features, represented in signals 418(1) and 418(2)). The wireless transceiver 440 wirelessly transmits the extracted body noise features and acoustic sound features to the local computing device 436 via a wireless link 441.

The local computing device 436 includes a wireless transceiver 442 and an activity classifier 422. The wireless transceiver 442 receives the extracted body noise features and acoustic sound features from the implantable component 434 via the wireless link 441. The extracted body noise features and acoustic sound features, again represented in signals 418(1) and 418(2), are provided to their activity classifier 420.

The activity classifier 420, which may be similar to activity classifier 120 described above with reference to FIGS. 1A and 1B, is configured to use the body noise features and acoustic sound features to classify the current or real-time activity of the recipient. That is, the activity classifier 420 is configured to use the signal features (i.e., characteristics) extracted from the signals 412 to generate a real-time classification of the detected body noises, where the classification corresponds to an associated current/real-time activity of the recipient (i.e., the activity of the recipient at the time the body noises within signals 412 is detected). A real-time activity classification determined by the activity classifier 420 is generally represented in FIG. 4 by arrow 422. In the example of FIG. 4, the activity classifier 420 provides the activity classification 422 to the wireless transceiver 442 for wireless transmission to the remote computing system 438.

The remote computing system 438 includes a wireless transceiver 444 and a logging and analytics module 424. The wireless transceiver 444 receives the activity classification 422 from the local computing device 436 via a wireless link 443. The wireless transceiver 444 provides the received activity classification 422 to the logging and analytics module 424. The logging and analytics module 424, which may be similar to logging and analytics module 124 described above with reference to FIGS. 1A and 1B, is configured to log (e.g., store) the activity classifications 422 generated for the recipient over time (e.g., one or days, one or more weeks, etc.) with time information. As noted above, the logging and analytics module 424 generates/populates, over time, an activity database 426 (i.e., the log of the activity classifications 122 over time). The activity database 426 may also be analyzed and used to generate one or more outputs 428.

As noted, FIG. 4 illustrates a body-noise lifestyle tracking system that includes a stand-alone implantable component in accordance with embodiments presented herein. FIGS. 5A and 5B illustrate an acoustic implant that includes components of a body-noise lifestyle tracking system, in accordance with embodiments presented herein.

More specifically, FIG. 5A is a schematic diagram illustrating an implantable middle ear prosthesis 550 in accordance with embodiments presented herein. The implantable middle ear prosthesis 550 is shown implanted in the head 551 of a recipient. FIG. 5B is a block diagram of the implantable middle ear prosthesis 502. For ease of description, FIGS. 5A and 5B will be described together.

Shown in FIG. 5A is an outer ear 501, a middle ear 502 and an inner ear 503 of the recipient. In a fully functional human hearing anatomy, the outer ear 1501 comprises an auricle 505 and an ear canal 506. Sound signals 507, sometimes referred to herein as acoustic sounds or sound waves, are collected by the auricle 505 and channeled into and through the ear canal 506. Disposed across the distal end of the ear canal 506 is a tympanic membrane 504 which vibrates in response to the sound signals (i.e., sound waves) 507. This vibration is coupled to the oval window or fenestra ovalis 552 through three bones of the middle ear 502, collectively referred to as the ossicular chain or ossicles 553 and comprising the malleus 554, the incus 556 and the stapes 558. The ossicles 553 of the middle ear 502 serve to filter and amplify the sound signals 507, causing oval window 552 to vibrate. Such vibration sets up waves of fluid motion within the cochlea 560 which, in turn, activates hair cells (not shown) that line the inside of the cochlea 560. Activation of these hair cells causes appropriate nerve impulses to be transferred through the spiral ganglion cells and the auditory nerve 561 to the brain (not shown), where they are perceived as sound.

As noted above, conductive hearing loss may be due to an impediment to the normal mechanical pathways that provide sound to the hair cells in the cochlea 560. One treatment for conductive hearing loss is the use of an implantable middle ear prosthesis, such as implantable middle ear prosthesis 550 shown in FIGS. 5A and 5B. The middle ear prosthesis 100 is, in general, configured to convert sound signals entering the recipient's outer ear 501 into mechanical vibrations that are directly or indirectly transferred to the cochlea 560, thereby causing generation of nerve impulses that result in the perception of the received sound.

The implantable middle ear prosthesis 550 includes implantable microphone 510(1), a main implantable component (implant body) 562, and an output transducer 568, all implanted in the head 125 of the recipient. The implantable microphone 510(1), main implantable component 562, and output transducer 124 can each include hermetically-sealed housings which, for ease of illustration, have been omitted from FIGS. 5A and 5B.

The main implantable component 562 comprises a processing module 564, a wireless transceiver 540, and a battery 565. The processing module 564 includes a body noises processor 516 and a sound processor 566.

In operation, the implantable microphone 510(1) is configured to detect input signals which include acoustic sound signals (sounds) and convert the sound signals into electrical signals 514 to evoke a hearing percept (i.e., enable the recipient to perceive the sound signals 507). More specifically, the sound processor 566 processes (e.g., adjusts amplifies, etc.) the received electrical signals 514(2) according to the hearing needs of the recipient. That is, the sound processor 566 converts the electrical signals 514(2) into processed signals 567. The processed signals 567 generated by the sound processor 566 are then provided to the output transducer 568 via a lead 569. The output transducer 568 is configured to convert the processed signals 567 into vibrations for delivery to hearing anatomy of the recipient.

In the embodiment of FIGS. 5A and 5B, the output transducer 568 is mechanically coupled to the stapes 558 via a coupling element 570. As such, the coupling element 570 relays the vibration generated by the output transducer 568 to the stapes 558 which, in turn, causes oval window 552 to vibrate. Such vibration of the oval window 552 sets up waves of fluid motion within the cochlea 560 which, in turn, activates hair cells (not shown) that line the inside of the cochlea 560. Activation of these hair cells causes appropriate nerve impulses to be transferred through the spiral ganglion cells and the auditory nerve 561 to the brain (not shown), where they are perceived as sound.

As noted above, the implantable middle ear prosthesis 550 is configured evoke perceptions of sound signals. Moreover, in accordance with embodiments presented herein, the implantable middle ear prosthesis 550 is further configured to capture the recipient's body noises for use in classifying the activity of the recipient. That is, the implantable middle ear prosthesis 550 is configured as a component of a body noise-based health monitoring system in accordance with embodiments presented herein.

More specifically, as shown in FIG. 5B, the implantable middle ear prosthesis 550 comprises the body noises processor 516. As noted, the microphone 510(1) is configured to detect input signals, which include acoustic sound signals (sounds). The input signals may also, in certain circumstances, include body noises, which, as a result, will be present in the electrical signals 514. In accordance with these examples, the electrical signals 514 are also provided to the body noises processor 516 in processing module 564.

The body noises processor 516, which may be similar to body noises processor 116 of FIGS. 1A and 1B, is configured to convert the electrical signals 514 into processed signals (not shown in FIGS. 5A and 5B) that represent the detected signals. That is, the body noises processor 516 outputs one or more processed signals representing features of the detected body noises (e.g., the body noises processor 516 extracts body noise features and acoustic sound features).

In the examples of FIGS. 5A and 5B, the wireless transceiver 540 wirelessly transmits the extracted body noise features and acoustic sound features to a computing device for further processing. For example, in certain embodiments, the implantable middle ear prosthesis 550 may be used with the local computing device 436 and the remote computing system 438 of FIG. 4 to form a body noise-based health monitoring system. In essence, the implantable middle ear prosthesis 550 replaces the implantable component 434 as the device that provides the body noise features and acoustic sound features for use in the activity classification.

FIG. 6 is a simplified schematic diagram illustrating an example spinal cord stimulator 650 that may form part of a body noise-based health monitoring system, in accordance with embodiments presented herein. The spinal cord stimulator 650 includes a microphone 610(1), a main implantable component (implant body) 662, and a stimulating assembly 676, all implanted in a recipient. The multi-channel sensor system 608 comprises a microphone 610(1) and an accelerometer 610(2).

The main implantable component 662 comprises a body noises processor 616, a wireless transceiver 640, a battery 665, and a stimulator unit 675. The stimulator unit 675 comprising, among other elements, one or more current sources on an integrated circuit (IC).

The stimulating assembly 676 is implanted in a recipient adjacent/proximate to the recipient's spinal cord 637 and comprises five (5) stimulation electrodes 674, referred to as stimulation electrodes 674(1)-674(5). The stimulation electrodes 674(1)-674(5) are disposed in an electrically-insulating carrier member 677 and are electrically connected to the stimulator 675 via conductors (not shown) that extend through the carrier member 677.

Following implantation, the stimulator unit 675 generate stimulation signals for delivery to the spinal cord 637 via stimulation electrodes 674(1)-674(5). Although not shown in FIG. 6, an external controller may also be provided to transmit signals through the recipient's skin/tissue to the stimulator unit 675 for control of the stimulation signals.

As noted above, the spinal cord stimulator 650 is configured to stimulate the spinal cord of the recipient. Moreover, in accordance with embodiments presented herein, spinal cord stimulator 650 is further configured to capture the recipient's body noises for use in classifying the activity of the recipient. That is, the spinal cord stimulator 650 is configured as a component of a body noise-based health monitoring system in accordance with embodiments presented herein.

More specifically, as shown in FIG. 6, the spinal cord stimulator 650 comprises the microphone 610(1) configured to capture/receive body noises. As shown, in the example of FIG. 1 the microphone 610(1) is mounted the spinal cord 637. The positioning of microphone 610(1) may be advantageous to detect body noises, but it is to be appreciated that this specific positioning is merely illustrative.

In operation, the microphone 610(1) converts detected input signals (e.g., body noises and/or external acoustic sounds, if present) into electrical signals (not shown in FIG. 6) which are provided to the body noises processor 616. The body noises processor 616, which may be similar to body noises processor 116 of FIGS. 1A and 1B, is configured to convert the electrical signals received from the microphone 610(1) into processed signals (not shown in FIG. 6) that represent the detected signals. That is, the body noises processor 616 outputs one or more processed signals representing features of the detected body noises (e.g., the body noises processor 616 extracts body noise features and acoustic sound features, if present).

In the examples of FIG. 6, the wireless transceiver 640 wirelessly transmits the extracted body noise features (and acoustic sound features, if present) to a computing device for further processing. For example, in certain embodiments, the spinal cord stimulator 650 may be used with the local computing device 436 and the remote computing system 438 of FIG. 4 to form a body noise-based health monitoring system. In essence, the spinal cord stimulator 650 replaces the implantable component 434 as the device that provides the body noise features and acoustic sound features for use in the activity classification.

As noted above, aspects of the techniques described herein are configured so as to protect the privacy of the individuals being monitored through the body noise-based health monitoring systems presented herein. In certain embodiments, these protections are provided by the body noises processors. For example, as noted above, the body noises processors presented herein may be configured to ensure that it is not possible for any captured speech to be reconstructed from the features. In another example, a federated learning approach could be used to protect a recipient's privacy.

In a federated learning approach, the activity classifiers for each individual/recipient operate and train independently using the body noise features and acoustic sound features extracted for the associated specific recipient. At certain points in time, the operational attributes (e.g., weights) for the different activity classifiers (e.g., machine learning algorithms) are provided to a centralized system (e.g., cloud computing system). The operational attributes from the different activity classifiers are then combined to form a federated activity classifier that is configured to improve the processing for all individuals. The federated activity classifier is then pushed down and instantiated for each of the individuals. This approach protects the individual's privacy in that none of the individual or recipient data (e.g., extracted body noise features and acoustic sound features) is provided to the centralized system. Instead only the operational attributes of the classifiers, which do not include any personal data, are provided to the centralized system (e.g., the data and training is local and just the machine learning weights are uploaded to the centralized system).

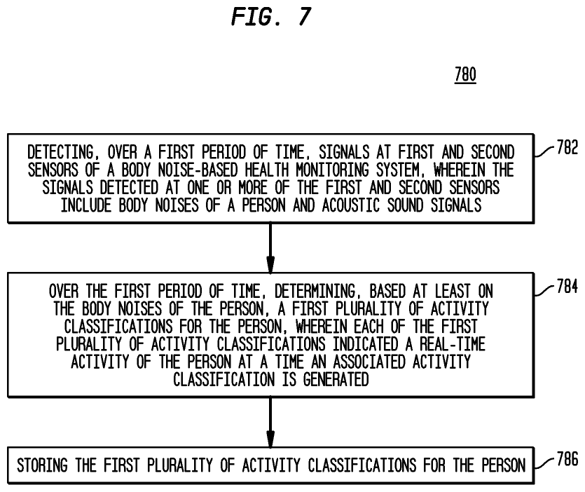

FIG. 7 is a flowchart of a method 780 in accordance with certain embodiments presented herein. Method 780 begins at 782 where, over a first period of time, first and second sensors of a body noise-based health monitoring system detect signals. The signals detected at one or more of the first and second sensors include body noises of a person and acoustic sound signals. At 784, over the first period of time, a first plurality of activity classifications for the person are determined based at least on the body noises of the person. Each of the first plurality of activity classifications indicates a real-time activity of the person at a time an associated activity classification is generated. At 786, the first plurality of activity classifications for the person are stored.

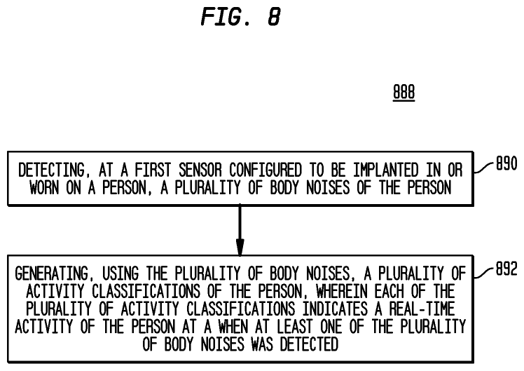

FIG. 8 is a flowchart of a method 888 in accordance with certain embodiments presented herein. Method 888 begins at 890 where a first sensor configured to be implanted in or worn on a person detects a plurality of body noises of the person. At 892, the plurality of body noises are used to generate a plurality of activity classifications of the person. Each of the plurality of activity classifications indicates a real-time activity of the person at a when at least one of the plurality of body noises was detected.

It is to be appreciated that the embodiments presented herein are not mutually exclusive.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A system, comprising:
at least a first sensor configured to be implanted in or worn on a person, wherein the at least first sensor is configured to detect body noises of the person; and
an activity classifier configured to determine, based at least on the body noises, an activity classification of a current activity of the person.

2. The system of claim 1, further comprising:
at least a second sensor configured to detect external acoustic sound signals that are associated with the signals of the first sensor; and
wherein the activity classifier configured to determine the person's current activity based at least on the body noises and the external acoustic sound signals associated with signals of the first sensor.

3. The system of claim 2, wherein the activity classifier is configured to:
over a first period of time, generate a first plurality of activity classifications for the person; and
over a second period of time, generate a second plurality of activity classifications for the person based on the body noises, the external acoustic sound signals associated with signals of the first sensor, and the first plurality of activity classifications generated over the first period of time.

4. The system of claim 1, wherein the activity classifier is configured to, over a first period of time, generate a first plurality of activity classifications for the person, and wherein the system further comprises:
a logging and analytics module configured to log the first plurality of activity classifications for the person.

5. The system of claim 4, wherein the logging and analytics module configured to log the first plurality of activity classifications with time information indicating at least one of a time-of-day or date when each of the first plurality of activity classifications was generated.

6. The system of claim 4, wherein the logging and analytics module configured to analyze the first plurality of activity classifications for the person and generate one or more baseline behavior patterns for the person.

7. The system of claim 6, wherein the activity classifier is configured to, over a second period of time, generate a second plurality of activity classifications for the person, and wherein the logging and analytics module is configured to:
analyze the second plurality of activity classifications generate one or more current behavior patterns; and
analyze the current behavior patterns relative to the one or more baseline behavior patterns to detect one or more differences between the one or more current behavior patterns and the one or more baseline behavior patterns.

8. The system of claim 7, wherein in response to detect one or more differences between one or more current behavior patterns and the one or more baseline behavior patterns, the logging and analytics module is configured to generate one or more messages configured to initiate or elicit a remedial action.

9. The system of claim 7, wherein based on the analyzing of the one or more current behavior patterns and the one or more baseline behavior patterns, the logging and analytics module is configured to generate one or more re-assurance messages.

10. The system of claim 2, wherein the first sensor is configured to generate a first electrical signal, and the second sensor is configured to generate a second electrical signal, and wherein the system comprises:
a body noises processor configured to extract features of the body noises and features of the external acoustic sound signals from the first and second electrical signals.

11. The system of claim 10, wherein the body noises processor is configured to extract one or more of time information, signal levels, frequency, or measures regarding a static and/or dynamic nature of the body noises and the external acoustic sound signals from the first and second electrical signals.

12. The system of claim 10, wherein the body noises processor is configured to extract features of the body noises and features of the external acoustic sound signals such that is not possible for any captured speech to be reconstructed from the features.

13. The system of claim 10, wherein the system is configured to one or more of prevent one or more activity classifications from being logged or hide one or more activity classifications from users other than the person.

14. A method, comprising:
detecting, over a first period of time, signals at first and second sensors of a body noise-based health monitoring system, wherein the signals detected at one or more of the first and second sensors include body noises of a person and acoustic sound signals;
over the first period of time, determining, based at least on the body noises of the person, a first plurality of activity classifications for the person, wherein each of the first plurality of activity classifications indicates a real-time activity of the person at a time an associated activity classification is generated; and
storing the first plurality of activity classifications for the person.

15. The method of claim 14, wherein storing the first plurality of activity classifications for the person comprises:
storing each of the first plurality of activity classifications with time information indicating at least one of a time-of-day or date when each of the first plurality of activity classifications was generated.

16. The method of claim 14, further comprising:
generating one or more baseline behavior patterns for the person based on the first plurality of activity classifications for the person.

17. The method of claim 16, further comprising:
detecting, over a second period of time, signals at the first and second sensors of a body noise-based health monitoring system;
over the second period of time, determining, based at least on the body noises of the person, a second plurality of activity classifications for the person;
generating one or more current behavior patterns for the person based on the second plurality of activity classifications; and
analyzing the one or more current behavior patterns relative to the one or more baseline behavior patterns to detect one or more differences between the one or more current behavior patterns and the one or more baseline behavior patterns.

18. The method of claim 17, wherein in response to detecting one or more differences between the one or more current behavior patterns and the one or more baseline behavior patterns, the method comprises:
generating one or more messages configured to initiate or elicit a remedial action.

19. The method of claim 16, further comprising:
receiving a plurality of auxiliary health inputs for the person from one or more auxiliary devices;
storing the plurality of auxiliary health inputs for the person; and
generating the one or more baseline behavior patterns for the person based on the plurality of auxiliary health inputs and the first plurality of activity classifications for the person.

20. The method of claim 14, wherein detecting, over the first period of time, the signals at first and second sensors include body noises and external acoustic sound signals associated with one or more of the body noises, and wherein the method comprises:
extracting features of the body noises and features of the external acoustic sound signals.

21. The method of claim 20, wherein the features of the body noises and features of the external acoustic sound signals comprise one or more of time information, signal levels, frequency, or measures regarding a static and/or dynamic nature of the body noises and the external acoustic sound signals.

22. The method of claim 14, wherein the first sensor and the second sensor are each configured to be implanted in the person.

23. A method, comprising:
detecting, at a first sensor configured to be implanted in or worn on a person, a plurality of body noises of the person; and
generating, using the plurality of body noises, a plurality of activity classifications of the person, wherein each of the plurality of activity classifications indicates a real-time activity of the person at a when at least one of the plurality of body noises was detected.

24. The method of claim 23, further comprising:
detecting, at a second sensor, acoustic sound signals received with one or more of the plurality of body noises;
extracting features of the plurality of body noises and the acoustic sound signals received with the one or more of the plurality of body noises; and
generating, using the features of the plurality of body noises and the features of the acoustic sound signals, a plurality of activity classifications of the person, wherein each of the plurality of activity classifications indicates a real-time activity of the person at a when at least one of the plurality of body noises was detected.

25. The method of claim 23, further comprising:
logging each of the plurality of activity classifications with time information indicating at least one of a time-of-day or date when each of the plurality of activity classifications was generated.

26. The method of claim 25, further comprising:
monitoring a health of the person based on the plurality of activity classifications logged with the time information.

27. The method of claim 26, wherein monitoring a health of the person based on the plurality of activity classifications logged with the time information comprises:
determining one or more baseline behavior patterns for the person based on a first subset of the plurality of activity classifications; and
detecting, based on a second subset of the plurality of activity classifications, one or more changes to the one or more baseline behavior patterns for the person.

28. The method of claim 27, wherein in response to detecting more changes to the one or more baseline behavior patterns comprises:
generating one or more messages configured to initiate or elicit a remedial action.

29. The method of claim 27, wherein monitoring a health of the person based on the plurality of activity classifications logged with the time information comprises:
generating one or more re-assurance messages.

30. The method of claim 23, further comprising:
receiving a plurality of auxiliary health inputs for the person from one or more auxiliary devices;
storing the plurality of auxiliary health inputs for the person; and
monitoring a health of the person based on the plurality of auxiliary health inputs and the plurality of activity classifications for the person.
